(19) United States
(12) Patent Application Publication
(10) Pub. No.: US 2018/0256853 A1
(43) Pub. Date: Sep. 13, 2018

(54) ADHESIVE CATHETER WITH DISINFECTANT AGENT

(71) Applicant: Kayla Kight, Waianae, HI (US)

(72) Inventor: Kayla Kight, Waianae, HI (US)

(21) Appl. No.: 15/919,759

(22) Filed: Mar. 13, 2018

Related U.S. Application Data
(60) Provisional application No. 62/470,618, filed on Mar. 13, 2017.

(51) Int. Cl. A61M 25/02; A61L 2/00

(52) U.S. Cl. CPC A61M 25/02; A61M 2025/0266; A61M 2025/0253; A61L 2/0088

(57) ABSTRACT

An adhesive device secures patient IVs and urinary Foleys on a patient's skin and releases alcohol via a tab element. In some embodiments the present invention comprises a flat dressing having and upper and a lower side, the lower side providing for a mixture of adhesive and disinfecting agent such as alcohol, the upper side having one or more catheter clips configured to receive and secure one or more IV, urinary Foley, or other such lines. The present invention may also provide for one or more tabs configured for use as a pulling site, wherein the tab may be peeled off, thereby exposing an alcohol membrane layer that may disinfect a portion of the patient's skin. In some embodiments, the present invention may provide for either an adult or a children's embodiment.

### CROSS-REFERENCE TO RELATED APPLICATION

This application claims priority to U.S. Provisional Application No. 62/470,618 filed on Mar. 13, 2017, the disclosure of which, including any materials incorporated by reference therein, is incorporated herein by reference in its entirety.

### TECHNICAL FIELD

The present invention relates generally to adhesive medical devices. More particularly, the present invention concerns an adhesive device that secures patient intravenous fluid lines (“IV's”) and urinary Foley catheters (“Foleys”) on a patient's skin and releases alcohol via a tab element.

### BACKGROUND

In a medical setting, “IVs” or intravenous therapies are both common and extremely important for delivering fluids and fluid-dissolved elements. Urinary Foleys, or Foley catheters for use in a patient's urinary tract, are also important and common elements of patient care. In their application, both IV lines and urinary Foley catheter lines need to be secured in place against the patient's body. This both ensures the patient's safety and enables a medical professional to work on the patient without the lines getting in the medical professional's way. One way these lines are secured against the patient's skin is through adhesive-mounted devices that stick to the patient's skin on one side and provide for holding elements on the other side that hold the lines in place.

The standard for how long IVs need to be changed is every four days unless another indication requires that the IV be changed sooner, such as infiltration or phlebitis. Unfortunately, a patient's skin can be damaged when an IV or Urinary Foley is changed or discontinued according to these or other protocols. In such a scenario, often a clinician rubs one or more alcohol nap wipes on top of the adhesive tape on top of the patient's skin, which can sometimes involve unintentional tearing of the patient's hair and skin, which in turn can potentially cause harm and introduce infection.

Attempts have been made, although unsuccessfully, to solve this problem. One illustrative attempt can be seen with respect to U.S. Pat. No. 5,092,323, incorporated by reference in its entirety herein, which generally discloses a moisture absorbing adhesive dressing configured to protect and cover an intravenous injection site. While this disclosure does provide for a versatile dressing, it fails to provide for a quick-release alcohol dispersion element.

Another attempt can be seen with respect to U.S. Pat. No. 4,921,199, incorporated by reference in its entirety herein, which generally discloses a device operable to assist a medical professional with inserting, managing, and removing an IV therapy and/or injection. However, this disclosure fails to provide for an adhesive-mounted device that can both secure IV and Foley lines as well as disperse alcohol with the pull of a tab.

Yet another attempt can be seen with respect to U.S. Pat. No. 4,726,716, incorporated by reference in its entirety herein, which generally discloses a Foley catheter and adhesive patch. While this disclosure does provide for adhesion of the Foley to a patient's skin, such as on the leg, it also fails to provide for alcohol distribution.

Various attempts have been made to solve the problems which may be found in the related art but have been unsuccessful. Therefore, a need exists for a new adhesive product that provides a disinfectant agent, such as an alcohol membrane embedded above the adhesive layer, and wherein the alcohol membrane is released when a tab is pulled, to avoid the challenges and problems with the prior art.

### SUMMARY OF THE INVENTION

It is to be understood that in the present disclosure, all embodiments are provided as illustrative and non-limiting representatives of many possible embodiments. In addition, the terms “is,” “can,” “will,” and the like are herein used as synonyms for and interchangeable with terms such as “may,” “may provide for,” and “it is contemplated that the present invention may” and so forth.

Furthermore, all elements listed by name, such as a catheter, adhesive patch, pull-tab, etc., are herein meant to include or encompass all equivalents for such elements. For example, in addition to a “pull tab”, any method of storage and quick release of a fluid onto a designated surface is also contemplated by the present invention. Such equivalents are contemplated for each element named in its particular herein. Also, while the invention will be described in connection with IV therapy lines and urinary Foley lines, it is understood that the invention is not limited in scope to use with IV therapy lines and urinary Foley lines, but may be used with other types of lines, fluids, adhesive elements, and in settings other than a medical setting. Such other types of lines, fluids, adhesive elements, and settings may include, by way of illustration and not limitation, PICC lines, Electrodes for ECGs, and other potential medical devices for temporarily securing to patient's skin for short-term and long-term durations.

For purposes of summarizing, certain aspects, advantages, and novel features of the present invention are provided herein. It is to be understood that not all such aspects, advantages, or novel features may be provided in any one particular embodiment. Thus, the disclosed subject matter may be embodied or carried out in a manner that achieves or optimizes one aspect, advantage, or novel feature or group of features without achieving all aspects, advantages, or novel features as may be taught or suggested.

In view of view of the foregoing disadvantages inherent in the known art, the present invention provides a novel solution for securing IV and urinary Foley lines to a patient's skin, as well as to disperse sanitizing alcohol to a controlled location via a pull-tab element. The general purpose of the present invention, which shall be described subsequently in greater detail, is to allow a user to secure IV and Foley lines, then change the lines and the dressings easily while also dispersing alcohol to the intended site, thereby avoiding infection and harm to the patient.

The features of the invention which are believed to be novel are particularly pointed out and distinctly claimed in the concluding portion of the specification. By way of non-limiting example, the present invention provides a novel solution for maintaining a user's body temperature. These and other features, aspects, and advantages of the present invention will become better understood with reference to the following drawings and detailed description.

In some embodiments, the present invention may provide for an adhesive layer mounted onto a plastic thin layer that is left unexposed until ready for skin use, an alcohol membrane layer, a pull tab, a securing locking device for IVs and urinary Foleys, all contained within a packaging that keeps out light and does not allow for the evaporation of the alcohol. In addition, the present invention may provide for an outer border that keeps the alcohol membrane layer intact, unable to evaporate, and ready for release with the pull-tab.

More specifically, the present invention comprises an adhesive device that secures patient IVs and urinary Foleys on a patient's skin and releases alcohol via a tab element. In various embodiments, the present invention provides for an adhesive dressing layer configured to lay flat against a patient's skin and having two sides. On the patient skin facing side, or the present invention's bottom side, the present invention may provide for an adhesive layer designed to make removable and sustained contact with a patient's skin. On the present invention's medical professional facing side, or upper side, the present invention may provide for one or more catheter clips that, when in use, secure one or more catheter, IV, or other fluid-bearing lines in place. The present invention may also provide for a tab element at the top of the present invention that a user may use to peel the present invention off of a patient's skin. It is contemplated that in some embodiments, the bottom side may provide for a combination adhesive and alcohol substance that, when applied, exposed, or otherwise activated, may either cause the present invention to adhere to a patient's skin, disinfect the patient's skin, or both.

In other embodiments, the present invention may be shaped like a bear face, cartoon shape, or other design. Such designs may make the present invention less scary to children, easier to use by medical professionals, serve both purposes, or cause a different effect.

Additionally, in some embodiments, the present invention's upper side may also provide for a surface that supports writing, wherein a user may write on the present invention's upper side such information as the date and time that the present invention was applied to a patient's skin.

Furthermore, in some embodiments, the present invention may provide for two or more tabs, which in some embodiments may be provided on opposite sides of the present invention.

Still further, in some embodiments, rather than being intermixed with the adhesive elements, the present invention may provide a simple pull-tab that releases an alcohol membrane layer that is designed to protect, disinfect and release the adhesiveness without harming the patient's skin.

Yet further, in other embodiments may provide that in addition to or instead of IV or urinary Foley lines, the present invention may be configured to receive and secure PICC lines, Electrodes for ECGs, and other lines or medical devices.

Even further, in some embodiments, the present invention may provide for adult versions and/or child versions, wherein the child version(s) may appear friendly like a sticker and may decrease the child's anxiety.

The unique features of this product will provide the following benefits for consumers everywhere: it may reduce waste of time and resources because no alcohol packets need be used. Clinicians may not have to rub alcohol wipes on top of the patient's skin vigorously. The patient's skin may remain intact. The present invention may decrease the risk for infection. The present invention may be less painful than alterative methodologies and devices.

The embodiments of the invention described herein are exemplary and numerous modifications, variations and rearrangements can be readily envisioned to achieve substantially equivalent results, all of which are intended to be embraced within the spirit and scope of the invention. Furthermore, while the preferred embodiment of the invention has been described in terms of the components and configurations, it is understood to that the invention is not intended to be limited to those specific dimensions or configurations but is to be accorded the full breadth and scope of the spirit of the invention.

### BRIEF DESCRIPTION OF THE DRAWINGS

These and other features, aspects and advantages of the present invention will become better understood with regard to the following description, appended claims, and accompanying figures where:

FIG. 1 shows a perspective view of an adhesive catheter with disinfectant agent in accordance with an embodiment of the invention.

FIG. 2 shows a perspective view of an alternative embodiment of an adhesive catheter with disinfectant agent in accordance with an embodiment of the invention.

FIG. 3 shows a perspective view of an alternative embodiment of an adhesive catheter with disinfectant agent in accordance with an embodiment of the invention.

FIG. 4 shows a perspective view of an alternative embodiment of an adhesive catheter with disinfectant agent in accordance with an embodiment of the invention.

FIG. 5 shows an angled top perspective view of an adhesive catheter with disinfectant agent in accordance with an embodiment of the invention.

FIG. 6 shows a perspective view of an adhesive catheter with disinfectant agent in accordance with an embodiment of the invention.

### DETAILED DESCRIPTION

The present invention overcomes the limitations of the prior art by providing a new and more effective self-cooling garment system and method of using the same.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any embodiment or element of an embodiment disclosed in this disclosure will be determined by its intended use.

It is to be understood that the drawings and the associated descriptions are provided to illustrate potential embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific details. Well-known features, elements or techniques may not be shown in detail in order not to obscure the embodiments.

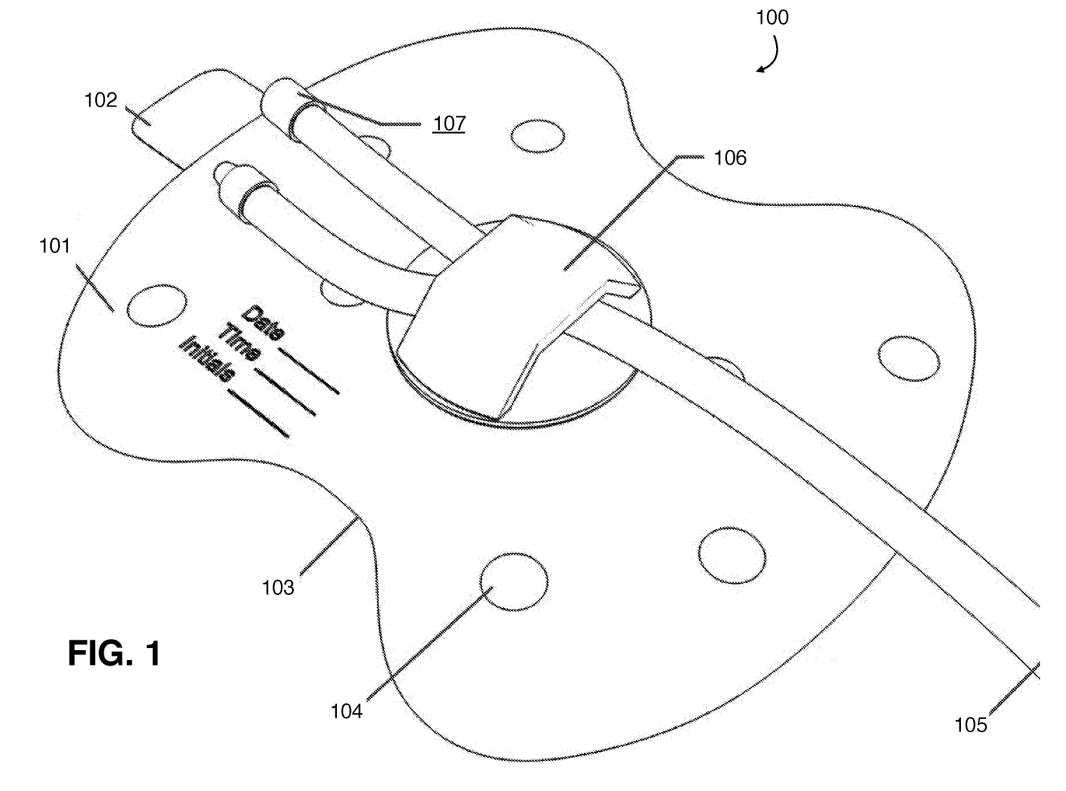

Turning attention to FIG. 1, a perspective view of an adhesive catheter with disinfectant agent in accordance with an embodiment of the invention is shown. In the embodiment depicted, a viewer may perceive adhesive catheter with disinfectant agent 100, adhesive dressing pad 101, pull tab 102, substrate layer with alcohol 103, holes 104, urinary catheter tube 105, catheter clip 106, and urinary catheter 107.

Adhesive dressing pad 101 may, in some embodiments, be a resilient, “spongy,” thin pad configured for use with wounds or abrasions. In some embodiments, adhesive dressing pad 101 may air-permeable from 0.01-100%. As may be disclosed elsewhere herein, adhesive dressing pad 101 may take any shape known in the art, such as but not limited to a figure-eight embodiment as shown in FIG. 1, and in some embodiments may generally be flat and thin. In addition to the elements disposed on, in, or through adhesive dressing pad 101, other elements such as lights, wireless or wired transponders, medical instruments, other pads, clips, surfaces configured to be written upon, thermometers, and so forth may also be attached to adhesive dressing pad 101.

Pull tab 102 may have at least two configurations with respect to the present invention. First, pull tab 102 may be configured as the point of contact between a person's hand and adhesive catheter with disinfectant agent 100, enabling the person to peel adhesive catheter with disinfectant agent 100 off of a surface, such as human skin. In such embodiments, pull tab 102 may be firmly affixed to adhesive dressing pad 101 or another element of adhesive catheter with disinfectant agent 100.

Alternatively, in such embodiments, pull tab 102 may be configured to be peelably or otherwise detachable from adhesive dressing pad 101 or another element of adhesive catheter with disinfectant agent 100. In such embodiments, pull tab 102 may serve to firmly connect to adhesive dressing pad 101 or another element of adhesive catheter with disinfectant agent 100 relative to one direction of force, but may be more easily detachable relative to another direction of force.

In other embodiments, pull tab 102 may be configured to release one or more portions of alcohol or other disinfectant when pulled. For example, in such embodiments, pull tab 102 may be attached to one or more sheets or layers of film, or may itself be a sheet, layer of film, wherein the layer of film or sheet prevents a portion of alcohol from being released. In such embodiments, pulling on pull tab 102 or peeling pull tab 102 off may remove the barrier and therefore release the alcohol or other disinfectant. In some embodiments, substrate layer with alcohol 103 may comprise the portion of alcohol or other disinfectant.

Consequently, in such embodiments, pull tab 102 may be comprised of any element known in the art, such as but not limited to plastic, elastomer, metal, wood, carbo fiber, or any other element.

In some embodiments, substrate layer with alcohol 103 may have at least one disinfectant agent, which in some embodiments may comprise a disinfectant alcohol. In other embodiments, substrate layer with alcohol 103 and/or its disinfectant agent may comprise an antibiotic cream, an antifungal, a pharmaceutically active compound, an astringent, a blend of herbal elements, a prescription cream, an antihistamine, an analgesic, a warming or cooling agent, or a combination of any such element, or other skin-permeable, skin-cleaning, or skin-cleansing agent.

Remaining with FIG. 1, in some embodiments, holes 104 may comprise one or more channels or pores in the surface of one or more elements of the present invention, including but not limited to adhesive dressing pad 101. The present invention may provide for any number or shape of holes 104, including but not limited to many perforations of very small size, or a hole 104 so large it extends entirely to the perimeter of adhesive dressing pad 101, and any combination of one or more holes 104 in between.

Urinary catheter tube 105 is shown in FIG. 1 as being relatively narrow and single, but those of skill in the art will recognize that urinary catheter tube 105 may comprise two or more urinary catheter tubes 105 that may be branched, wider or narrower, selectively connected to one or more common liquid flows, and so forth. Although typically comprised of medical grade plastic, a biodegradable element may comprise urinary catheter tube 105 in some embodiments, as well as a tube configured with thermochromic or other selectively illuminating elements.

Catheter clip 106, in some embodiments, may be configured to contain one or more urinary catheters 107. In some embodiments, catheter clip 106 may be configured to contain multiple urinary catheter 107 and may provide one or more points of attachment for one or more of the urinary catheter 107 contained thereon. As well, catheter clip 106 may provide for any attachment element or means for attachment known in the art, such as a clip (shown), a strap, an elastic band, a hook and loop configuration, magnetic attachment elements, snap enclosures, and any other

Urinary catheter 107 is also shown in FIG. 1 as being relatively narrow and single, but those of skill in the art will recognize that urinary catheter 107 may comprise two or more urinary catheters 107 that may be branched, wider or narrower, selectively connected to one or more common liquid flows, and so forth. Although typically comprised of medical grade plastic, a biodegradable element may comprise urinary catheter 107 in some embodiments, as well as a tube configured with thermochromic or other selectively illuminating elements.

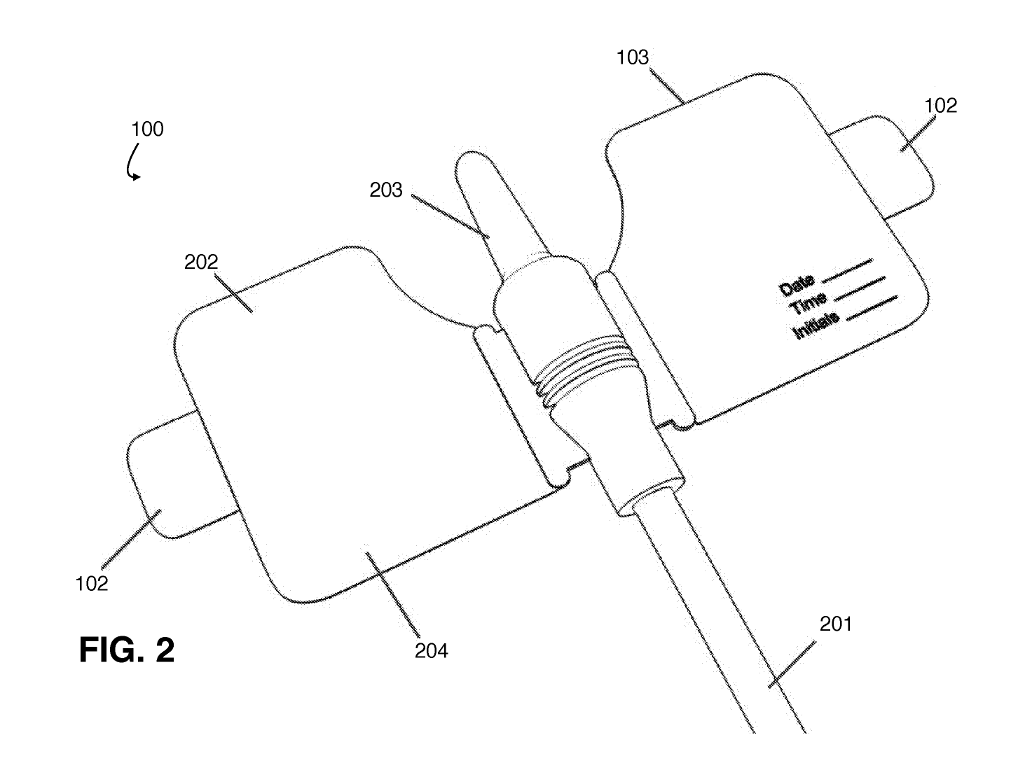

With respect to FIG. 2, a perspective view of an alternative embodiment of an adhesive catheter with disinfectant agent in accordance with an embodiment of the invention may be perceived. In the embodiment depicted, peripheral intravenous line (PIV) 201, PIV adhesive dressing pad 202, IV tube 203, and PIV adhesive pad surface 204, are shown, along with pull tab 102, substrate layer with alcohol 103.

As may be generally known in the art, peripheral intravenous line (PIV) 201 here generally is offered as an alternative to urinary catheter tube 105, but in some embodiments, both peripheral intravenous line (PIV) 201 and urinary catheter tube 105 may be provided on the same adhesive pad, whether PIV adhesive dressing pad 202, adhesive dressing pad 101, or a different type of adhesive dressing.

Relatedly, IV tube 203 here is configured for use with peripheral intravenous line (PIV) 201 and PIV adhesive dressing pad 202, and as an alternative to urinary catheter 107, but in some embodiments, both IV tube 203 and urinary catheter 107 may be provided on the same adhesive pad, whether PIV adhesive dressing pad 202, adhesive dressing pad 101, or a different type of adhesive dressing.

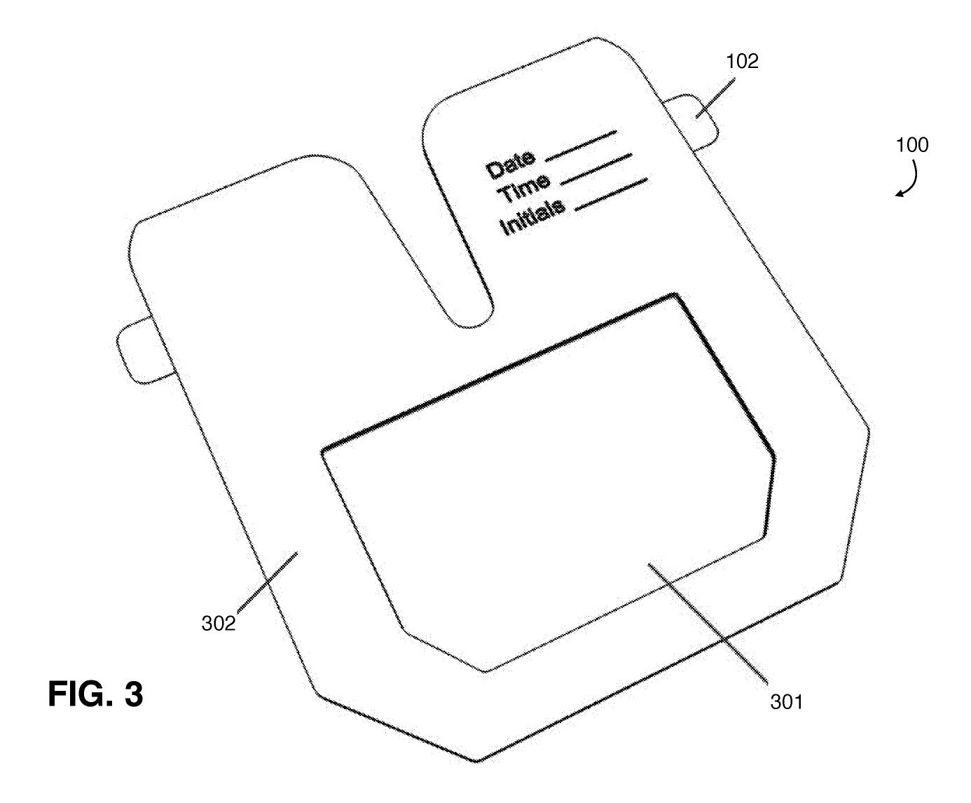

Moving attention to FIG. 3, a perspective view of an alternative embodiment of an adhesive catheter with disinfectant agent in accordance with an embodiment of the invention is shown. In the embodiment depicted, a viewer may perceive clear bio occlusive dressing with window 301, alternative adhesive dressing pad configuration 302, and pull tab 102. Clear bio occlusive dressing with window 301 may be of any configuration known in the art, including comprising one or more viewing areas, and occupying more or less of alternative adhesive dressing pad configuration 302.

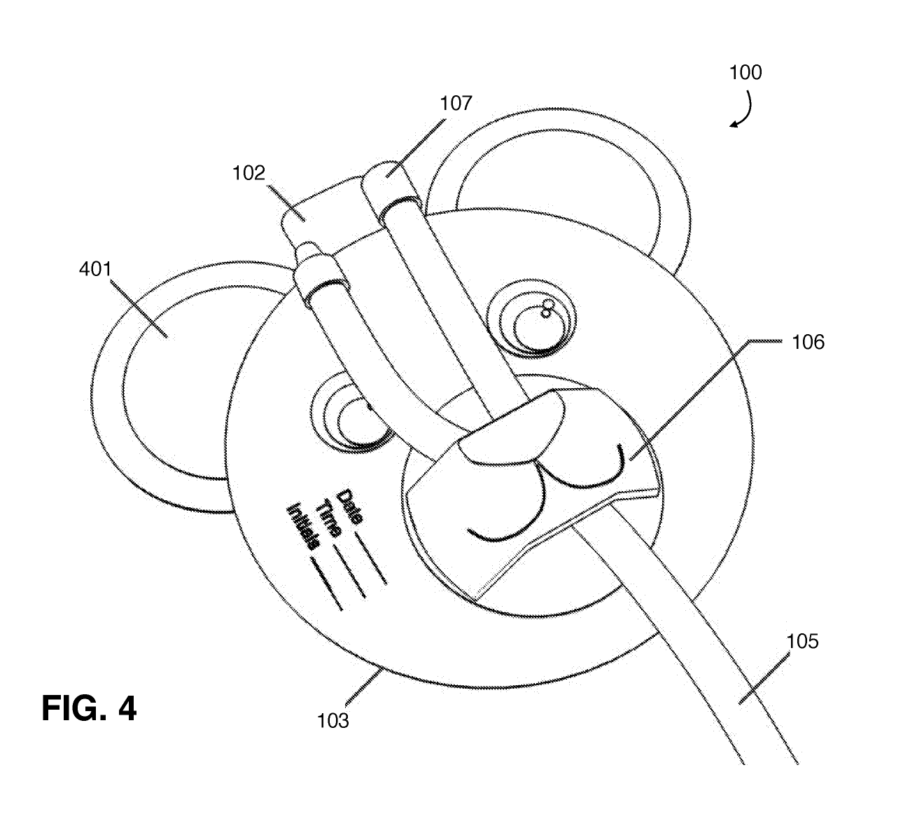

Turning attention to FIG. 4, a perspective view of an alternative embodiment of an adhesive catheter with disinfectant agent in accordance with an embodiment of the invention may be perceived. In the embodiment depicted, children's configuration of adhesive dressing pad 401 is shown, along with pull tab 102, substrate layer with alcohol 103, urinary catheter tube 105, catheter clip 106, and urinary catheter 107.

As may be generally perceived in FIG. 4, children's configuration of adhesive dressing pad 401 is shown as a monkey face, but any other any shape appealing to children, such as but not limited to any animal or element, such as an elephant, hippo, lion, bear, fish, bug, giraffe, frog, bunny, flower, rainbow, sun, moon, star, tree, fire truck, dump truck, princess, magic wand, train, and so forth is contemplated as shape(s) for children's configuration of adhesive dressing pad 401.

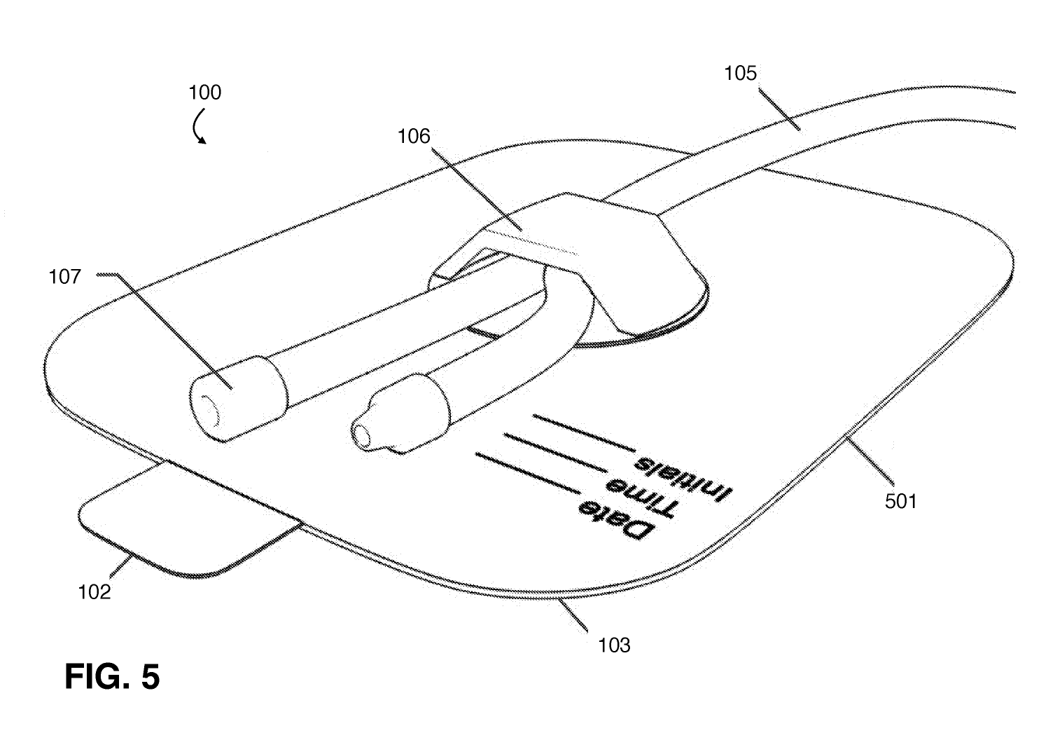

With respect to FIG. 5, an angled top perspective view of an adhesive catheter with disinfectant agent in accordance with an embodiment of the invention is depicted. In the embodiment of FIG. 5, a viewer may perceive rectangle embodiment of adhesive dressing pad 501, along with pull tab 102, substrate layer with alcohol 103, urinary catheter tube 105, catheter clip 106, and urinary catheter 107.

Although rectangle embodiment of adhesive dressing pad 501 is shown in FIG. 5 as having rounded edges, any shape of rectangle, square, circle, oval, or other shape is contemplated for any configuration of adhesive dressing by the present invention, including but not limited to adhesive dressing pad 501, adhesive dressing pad 101, PIV adhesive dressing pad 202, alternative adhesive dressing pad configuration 302, and children's configuration of adhesive dressing pad 401.

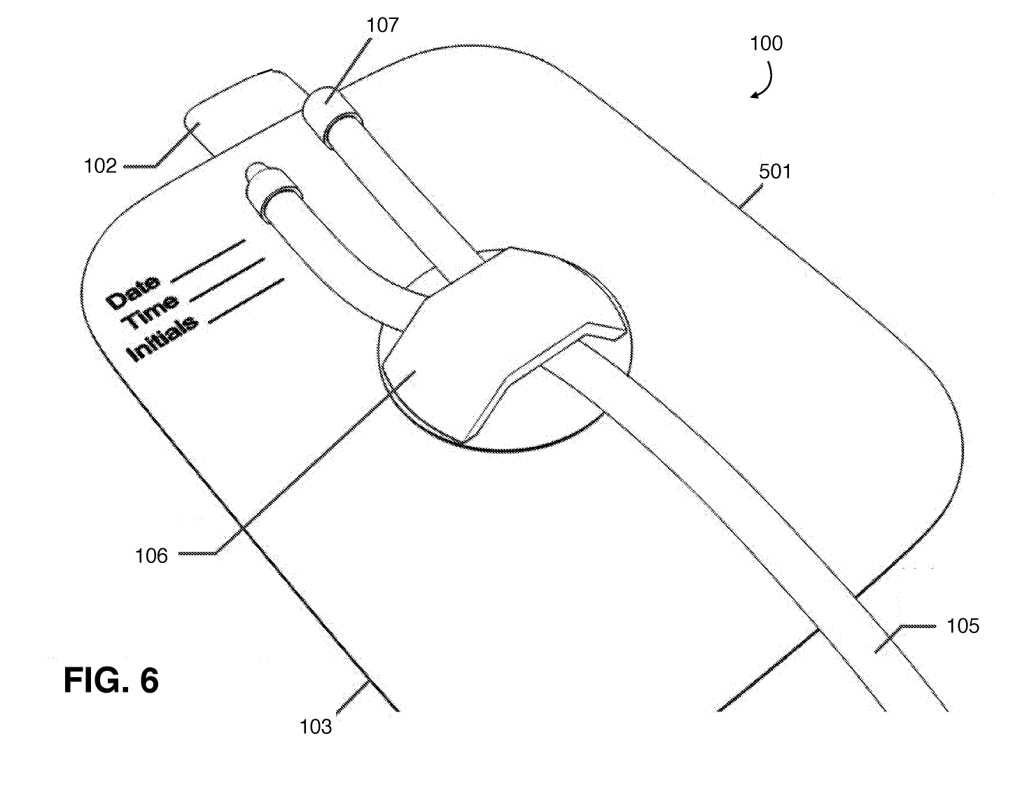

Moving to FIG. 6, a perspective view of an adhesive catheter with disinfectant agent in accordance with an embodiment of the invention is shown. In the embodiment depicted, rectangle embodiment of adhesive dressing pad 501, pull tab 102, substrate layer with alcohol 103, urinary catheter tube 105, catheter clip 106, and urinary catheter 107 may be perceived.

### Conclusions, Ramifications, and Scope

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

While the present invention generally described herein has been disclosed in connection with a number of embodiments shown and described in detail, various modifications should be readily apparent to those of skill in the art.

What is claimed is:

1. An adhesive catheter, comprising:
at least one adhesive dressing pad having a top side, an upper surface, a bottom side, a bottom surface, at least one edge;
at least one adhesive disinfectant substrate having at least one disinfectant agent, wherein the at least one adhesive disinfectant substrate is disposed on the bottom surface;
at least one pull tab disposed on the bottom surface;
at least one tube clip disposed on the at least one upper surface, wherein the at least one tube clip is configured to removably contain at least one tube; and
at least one tube held in place by the at least one at least one tube clip.

2. The adhesive catheter of claim 1, further comprising at least one aperture configured to transverse the at least one adhesive dressing from the top side to the bottom side.

3. The adhesive catheter of claim 1, wherein the at least one at least one tube clip is at least one catheter clip and the at least one tube is at least one catheter.

4. The adhesive catheter of claim 1, further comprising at least one peripheral intravenous line, wherein the at least one at least one tube clip is at least one peripheral intravenous clip and the at least one tube is at least one peripheral intravenous line.

5. The adhesive catheter of claim 4, wherein the at least one at least one tube clip is at least one peripheral intravenous clip and at least one catheter clip, and wherein the at least one tube is at least one peripheral intravenous line and at least one catheter.

6. The adhesive catheter of claim 1, wherein the at least one at least one adhesive dressing pad is configured as a rectangle with rounded corners.

7. The adhesive catheter of claim 1, wherein the at least one at least one adhesive dressing pad is configured as a figure eight.

8. The adhesive catheter of claim 1, wherein the at least one at least one adhesive dressing pad is configured in the shape that appeals to children.

9. The adhesive catheter of claim 1, wherein the at least one at least one adhesive dressing pad is configured in a butterfly shape.

10. The adhesive catheter of claim 1, further comprising at least one clear bio occlusive window disposed on the upper surface.

11. The adhesive catheter of claim 1, wherein the at least one pull tab is at least two pull tabs.

12. The adhesive catheter of claim 1 further comprising at least one removable barrier attached to the at least one pull tab, wherein the at least one barrier at least partially covers the at least one adhesive disinfectant substrate, and wherein the at least one pull tab is removable.

13. The adhesive catheter of claim 1, wherein the at least one disinfectant agent is alcohol.

14. The adhesive catheter of claim 1, wherein the at least one disinfectant agent is an antibiotic cream.

15. The adhesive catheter of claim 1, wherein the at least one disinfectant agent is an antihistamine cream.

16. The adhesive catheter of claim 1, wherein the at least one disinfectant agent is at least one pharmaceutically active compound.
